(19) United States
(12) Patent Application Publication
(10) Pub. No.: US 2017/0266362 A1
(43) Pub. Date: Sep. 21, 2017

(54) SYSTEM FOR REMOVAL OF PRO-INFLAMMATORY MEDIATORS AS WELL AS GRANULOCYTES AND MONOCYTES FROM BLOOD

(71) Applicant: 3M INNOVATIVE PROPERTIES COMPANY, St. Paul, MN (US)

(72) Inventors: Bodo von Harten, Wuppertal (DE); Detlef Krieter, Wurzbug (DE); Horst-Dieter Lemke, Obernburg (DE)

(21) Appl. No.: 15/505,724

(22) PCT Filed: Aug. 25, 2015
(86) PCT No.: PCT/EP2015/069415

§ 371 Date: Feb. 22, 2017

(30) Foreign Application Priority Data
Aug. 26, 2014 (EP) 14182261.9

(51) Int. Cl. A61M 1/36

(52) U.S. Cl. CPC A61M 1/3633; A61M 1/3679; A61M 1/3472

(57) ABSTRACT

A blood treatment system comprising at least one first device and at least one second device, wherein the first device is a membrane filter for the removal of toxic mediators from blood and the second device is suitable for the removal of granulocytes and monocytes from blood. The first device has a first blood flow path a first blood flow path for conducting blood through and the second device has a second blood flow path. The first and second devices are serially connected in succession in such a way that the first blood flow path is in fluid communication with the second blood flow path.

The membrane has an interior filter space in its housing and a semipermeable membrane arranged in the interior filter space, which membrane divides the interior filter space into a retentate chamber and permeate chamber. The housing has a blood inlet device and a blood outlet device that are in fluid communication with the retentate chamber, as well as a permeate outlet for diverting permeate from the permeate chamber. The blood inlet device, the retentate chamber and the blood outlet device form the first blood flow path. The membrane filter has a separation characteristic such that the sieve coefficient for albumin, SK_{Alb}, is within the range from 0.015 to 0.35.

The present invention relates to a system for the treatment of blood, in particular for the treatment of sepsis.

Sepsis and systemic inflammatory reactions (SIRS) are the most frequent cause of death in intensive care units, with mortality rates between 30% and 70%. Sepsis is a disease that is characterized by a complex systemic inflammatory reaction of the organism to the ingress of infectious agents. The inflammatory response leads to organ dysfunction of varying degrees and often ends with the death of the patient. Affected patients therefore die less from the direct effects of the bacterial infection, but instead primarily from the systemic effects of the often excessive inflammatory response of the body. The control of this immune response, in which the production of what are known as proinflammatory mediators such as cytokines by neutrophilic granulocytes and monocytes plays a key role, becomes increasingly difficult to control with progression of sepsis.

The administration of antibiotics and supportive measures, i.e. the administration of circulation-assisting drugs, artificial respiration and kidney replacement therapy, are established in the standard therapy for sepsis. By contrast, the additional administration of cytokine-inhibiting substances has proven ineffective. Beyond that, in recent years several concepts for the removal of cytokines have been presented, by extracorporeal hemofiltration on the one hand and by extracorporeal adsorption on the other.

Concepts for the removal of cytokines or generally proinflammatory mediators by extracorporeal hemofiltration are described for example in U.S. Pat. No. 5,571,418, US2012/0312732, EP-A 2 281 625, WO 03/009885 or WO 2011/131534. The extracorporeal adsorption of cytokines is, for example, the subject of DE-A 199 13 707, WO 2013/025483, WO2012/094565 or US 2013/0011824.

Finally, methods and devices for its implementation are described in which cytokines are separated from the blood via membrane filters in the form of, for example, ultrafilters or plasma filters with the permeate, and in which the permeate containing the cytokines is channeled through an adsorber for the specific adsorption of cytokines, and finally the permeate purified in this way is returned to the patient (see for example WO 00/02603, WO03/009885, US2012/0312732, EP-A-0 787 500, EP-A-0 958 839).

Experiments in a septic animal model point to a positive effect of the cytokine filtration (in a pig) or cytokine adsorption (in a rat). However, initial studies on humans have thus far been unable to confirm this, even though it was possible to prove an effective reduction of the cytokine concentrations in the blood.

The disadvantage of the pure cytokine removal by an extracorporeal method—whether by filtration or adsorption—is that inflammatorily activated, cytokine-generating leukocytes are not eliminated in this way, and their persistent excessive immune response—and thus the disease process—continue.

WO 2007/025735 deals with the treatment of sepsis and follows the approach that activated leukocytes contribute to the production of cytokines, which in turn activate other immune cells, which ultimately leads to the systemic overreaction of the immune defense system and to sepsis. For the treatment of sepsis, WO 2007/025735 therefore proposes a filter for the removal of activated leukocytes in which blood flows through a filter material in what is known as a dead end mode, in which the leukocytes are held back and thereby removed from the blood. In one embodiment, the filter material can be provided with ligands or other bioactive substances that, for example, specifically interact with cytokines and cause an additional removal of cytokines in the flow of blood to be treated by the filter material.

The object of the present invention is to provide a system for the treatment of blood by means of which patients suffering in particular from sepsis can be effectively treated.

The object is achieved by a blood treatment system comprising at least one first device and at least one second device,
wherein the at least one first device has a first blood flow path for channeling blood, and the at least one second device has a second blood flow path for channeling blood, and wherein the at least one first device and the at least one second device are switched serially in succession in such a way that the first blood flow path is in fluid communication with the second blood flow path,
wherein the at least one first device is a membrane filter for the removal of toxic mediators from blood, and the at least one second device is suitable for the removal of granulocytes and monocytes from blood, and
where the membrane filter has a housing, an interior filter space formed in the housing, and a semipermeable membrane arranged in the interior filter space that divides the interior filter space into a retentate chamber and permeate chamber,
wherein the housing has a blood inlet device and a blood outlet device that are in fluid communication with the retentate chamber, and the blood inlet device, the retentate chamber and the blood outlet device form the first blood flow path for channeling blood through the first device, and
wherein the housing further has a permeate outlet for diverting permeate passing through the semipermeable membrane from the permeate chamber and
wherein the membrane filter has a separation characteristic such that the sieve coefficient for albumin SK_{Alb} is within a range from 0.015 to 0.35.

In the application, a cascading (i.e. successive) treatment of the blood flowing through the system and to be treated is done with the blood treatment system according to the invention, in which proinflammatory mediators such as cytokines are removed in the at least one device, and granulocytes and monocytes are removed in the second device. Thus, the blood can pass through the system according to the invention in such a way that it first flows through the at least one device—and, as a result, the proinflammatory mediators are removed first—and then through the at least one second device for the removal of granulocytes and monocytes. It likewise is possible that the blood passes through the system according to the invention in such a way that it first flows through the at least one second device—wherein the granulocytes and monocytes are thus removed first—and then through the at least one device for the removal of the proinflammatory mediators.

The at least one first and at least one second device are connected to each other in such a way that the first blood flow path for the channeling of blood of the first device is in fluid communication with the second blood flow path of the second device. This results in the blood introduced into the system being treated as it passes through the at least one first and at least one second device in succession in two steps independent of one another. The processes of removing proinflammatory mediators and removing granulocytes and monocytes are decoupled from each other and each are performed on the blood itself. The decoupling has the advantage that the individual devices can be specifically oriented to the respective removal process.

Depending on the application, it is possible that the system for blood treatment comprises a single first device and a single second device whose bloodflow paths are connected to each other. However, it is also possible that, for example, a single first device is connected to two second devices connected in parallel to each other so that the retentate exiting the first device and purified of proinflammatory mediators is divided into two substance flows and is supplied to the second blood flow paths of the two second devices. It is also possible that the system for blood treatment comprises a single first device and a single second device, and that the retentate exiting the first device is divided into two substance flows, wherein the one substance flow flows through the single second device and the second substance flow is guided into the bypass without further treatment. The substance flow that exits the second device and is purified of granulocytes and monocytes and the substance flow conducted within the bypass can then be merged after the second device (viewed in the direction of flow) and conducted as a total flow, for example to the patient to be treated. Of course, other combinations of single or multiple first and second devices are possible in the system according to the invention for blood treatment.

Within the membrane filter, the blood to be treated flows in the first blood flow path via the blood inlet device into the interior filter space, and through the retentate chamber on the retentate side of the semipermeable membrane. In the flow through the retentate chamber, a portion of the blood to be treated passes through the semipermeable membrane as permeate or ultrafiltrate, wherein the semipermeable membrane is designed with regard to its size in such a way that the pro-inflammatory mediators to be removed from the blood can be transported as a portion of the permeate or ultrafiltrate through the pores into the permeate chamber. The blood treated in this way and enriched with proinflammatory mediators exits the membrane filter on the first flow path via the blood outlet device, whereas the permeate containing the proinflammatory mediators removed from the blood exits the membrane filter via the permeate outlet.

In a preferred embodiment, the semipermeable membrane is at least one hollow fiber membrane having a wall and a lumen surrounded by the wall. In a preferred embodiment, the at least one hollow fiber membrane can, via its wall, have an asymmetric pore structure with a separating layer on the side of the wall of the hollow fiber membrane facing the lumen. Especially preferably, a plurality of hollow fiber membranes is arranged as a bundle within the membrane filter. In an especially preferred embodiment, the retentate chamber of the membrane filter is formed by the lumen of the at least one hollow fiber membrane.

The semipermeable membrane of the membrane filter is preferably a hydrophilic membrane. In this context, an especially preferred embodiment of the hydrophilic membrane is made of a hydrophobic first polymer that is combined with a hydrophilic second polymer. Possibilities for the first polymer include technical plastics from the group of aromatic sulfonated polymers, such as polysulfone, polyethersulfone, polyphenylenesulfone or polyarylethersulfone, the polycarbonates, polyimides, polyetherimides, polyetherketones, polyphenylene sulfides, copolymers or modifications of these polymers or mixtures of these polymers. In an especially preferred embodiment, the hydrophobic first polymer is a polysulfone or a polyethersulfone having the repeating molecular units illustrated in formulas (I) and (II) below

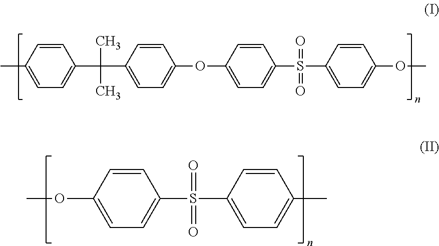

Long-chain polymers that, on the one hand, have a compatibility with the synthetic first polymer and that have repeating polymer units which are themselves hydrophilic are advantageously used as a hydrophilic second polymer. Preferably, the hydrophilic second polymer is polyvinylpyrrolidone, polyethylene glycol, polyvinyl alcohol, polyglycolmonoester, polysorbitate, such as polyoxyethylene sorbitan monooleate, carboxylmethyl cellulose, or a modification or copolymer of these polymers. Polyvinylpyrrolidone is especially preferable.

The membrane filter for the removal of proinflammatory mediators may, for example, have the form of standard hemofilters in which the supply and the discharge of the blood to be treated is done via a blood inlet device or a blood outlet device in the end caps of the membrane filter, which are in fluid communication with the lumina of the hollow fiber membranes arranged in a bundle in the membrane filter, which lumina form the retentate chamber. At least one outlet device—that is, the permeate outlet through which the permeate that contains the proinflammatory mediators removed from the blood exits the membrane filter—usually feeds via the wall of the housing into the outer space around the hollow fiber membranes, that is, into the permeate or filtrate chamber. However, as far as the semipermeable membrane contained therein is concerned, the membrane filter according to the invention differs from the typical hemofilters, as explained below.

In the at least one first device—that is, in the membrane filter for the removal of toxic mediators from blood—in the application a portion of the plasma water is removed as ultrafiltrate from the blood flowing on the retentate side, wherein the ultrafiltrate contains the toxic mediators contained in the blood which, because of their molecular size (in what is known as the medium molecular range), can pass through the semipermeable membrane because of the separation characteristic of the membrane. By contrast, important components of the blood such as cellular components, larger proteins dissolved in the blood plasma such as albumin, immunoglobulins, HDL or LDL, antibodies or fibrinogens are to an overwhelming degree or nearly completely held back by the semipermeable membrane contained in the membrane filter. The membrane filter according to the invention that contains this semipermeable membrane has, according to the invention, a sieve coefficient for albumin in blood SK_{Alb} within the range from 0.015 to 0.35. For the membrane filter, the sieve coefficient for albumin in blood SK_{Alb} is preferably within the range from 0.05 to 0.3, and especially preferably within the range from 0.1 to 0.25. The semipermeable membrane of the membrane filter according to the invention thus lets through certain proportions of albumin to which toxic mediators can be bonded at that time. Membrane filters according to the invention having sieve coefficients of this type exhibit a separation limit in the range from 50,000 to 150,000 daltons.

In another preferred embodiment, immunoglobulin G (IgG) having a molecular weight of approx. 180,000 daltons is nearly completely retained in the membrane filter or by the semipermeable membrane of the membrane filter. The membrane filter preferably has a sieve coefficient for IgG SK_{IgG} within the range from 0.001 to 0.1. Especially preferably, the sieve coefficient for IgG SK_{IgG} is within the range from 0.003 to 0.08.

The semipermeable membrane contained within the membrane filter according to the invention, or the membrane filter according to the invention, therefore differs from plasma filters which are frequently used in the field of blood purification, which have a separation limit exceeding approximately two million daltons, and in which a nearly complete separation of the aforementioned components dissolved in the blood plasma from the blood cells occurs via a separation of blood plasma. The plasma filtration membranes contained in such plasma filters thus have a much more open structure than the membranes of the membrane filter according to the invention. This more open structure simultaneously results in high permeabilities of the plasma filtration membranes, resulting in ultrafiltration rates for water, UFR_{water}, in excess of approx. 15,000 ml/(h m² mmHg).

On the other hand, the membrane filter according to the invention or the semipermeable membrane contained therein differs from hemodialyzers, hemodiafilters or hemofilters, or the membranes used therein, whose separation limit—at up to 40,000 daltons in whole blood—is designed in such a way that they at least nearly completely retain albumin and molecules larger than albumin, and for which sieve coefficients for albumin in blood SK_{Alb} of less than 0.005 are realized.

Preferably, the semipermeable membrane of the present membrane filter has an ultrafiltration rate in water or hydraulic permeability UFR_{water} within the range from 500 to 2000 ml/(h m² mmHg). Especially preferably, the hydraulic permeability is within the range from 500 to 1500 ml/(h m² mmHg). Most suitable is a semipermeable membrane having a UFR_{water} with the range from 800 to 1200 ml/(h m² mmHg). In this way, a sufficiently fast removal of toxic mediators during the blood treatment is achieved.

In the application, the permeate or ultrafiltrate—which contains the pro-inflammatory mediators removed from the blood—produced in the membrane filter can be discarded. However, it is also possible to purify the permeate or ultrafiltrate—that is, to remove the proinflammatory mediators from the permeate, for example via suitable adsorbers—and to return the purified permeate to the treated flow of blood.

Centrifuge devices, for example, can be used as a second device that is designed and suitable for the removal of granulocytes and monocytes. However, the at least one second device for the removal of granulocytes and monocytes is preferably a filter, an adsorber, or a combination of the two which are designed and suitable for the removal of granulocytes and monocytes.

In a preferred embodiment, the at least one second device can be a filter for the removal of granulocytes and monocytes, with a filter housing that has an interior space as well as an inlet device and an outlet device that are in fluid communication with the interior space,
wherein a filter material having flow channels, through which flow channels blood may flow, is arranged in the interior space of the filter housing,
wherein inlet device, outlet device and flow channels of the filter material form the second flow path within the interior space, and
wherein the filter material for the separation of granulocytes and monocytes is adapted by size exclusion and/or adsorption.

The filter material can be a fibrous material, for example a non-woven material, or a material in the form of one or more layers of fabric. Such filters are described in, for example, EP-A 0 155 003, EP-A 1 444 996, EP-A 1 553 113, EP-A 1 582 228, EP-A 1 754 496, US 2011/0031191, WO 2004/018078, WO 2004/039474, WO 2005/002647 or WO 2006/061862. In the filters disclosed in these documents, a removal via specific interactions between the filter material and the granulocytes and monocytes—that is, via adsorption—can also occur in the filter materials used, in addition to or instead of a removal of granulocytes and monocytes via size exclusion. Filters are also known having filter materials whose surface properties are designed for the adsorption of leukocytes, granulocytes or monocytes (see for example: EP-A 0 478 914, EP-A 0 606 646, EP-A 1 016 426, U.S. Pat. No. 4,476,023, WO 2004/064980, WO 2008/028807). Such filters for the removal of leukocytes, granulocytes and monocytes are also commercially available under the trade name Cellsorba™ (Asahi Medical Co. Ltd.).

The filter material can also be present in the form of porous materials through which can flow blood from which leukocytes (for example) are to be removed. Porous filter materials in the form of semipermeable membranes are disclosed, for example, in EP-A 0 606 646, EP-A 1 666 129 or U.S. Pat. No. 5,478,470.

In another preferred embodiment of the system for the treatment of blood, the at least one second device for the removal of granulocytes and monocytes from blood can be an adsorber with a housing which has an inner side surrounding an interior space, as well as an inlet device and an outlet device,
wherein a plurality of threads is arranged in the interior space,
wherein the threads have at least one of their ends embedded in a sealing compound that is joined to the housing inner side in such a way that an outer space through which blood can flow around the threads is formed that is in fluid communication with the inlet device and the outlet device, whereby inlet device, outlet device and interior space form the second flow path,
wherein the arrangement of the threads has a high degree of order, wherein a high degree of order is understood to mean that a proportion of at least 25% of the threads is arranged along their length direction, and
wherein the threads based on organic polymers cause formation of the complement activation product C5a in a concentration of at least 10 μg per m² of thread surface.

Preferably, the threads in these devices are hollow threads with a lumen and a wall surrounding the lumen as well as an interior, lumen-side surface and an outer surface, wherein the hollow threads are arranged in the housing in such a way that only the outer surfaces of the hollow threads are accessible for the blood flowing through the outer space, but the lumina of the hollow threads, by contrast, are not accessible for a fluid.

Such adsorbers for the removal of leukocytes, such as granulocytes and monocytes, are described in US 2008/0203024 and US 2010/0084331, the disclosure of which is explicitly referred to at this point.

In another preferred embodiment, the at least one second device for the removal of granulocytes and monocytes from blood can be an adsorber with a housing that has an inner side surrounding an interior space as well as an inlet device and an outlet device, wherein an adsorption material made up of particles is arranged in the interior space. An outer space through which blood can flow is formed around the particles of the adsorption material, which outer space is in fluid communication with the inlet device and the outlet device, whereby inlet device, outlet device and interior space form the second flow path. In these adsorbers, in the application the blood flows on the second blood flow path via the intake device into the interior space, flows around the adsorber particles, and exits the interior space via the outlet device.

Preferably, particles based on cellulose acetate or styrene are used as adsorber particles in the adsorbers. However, other materials—such as polyamides, polyethylene terephthalate or polyacylnitrile—are known that were subjected to a surface modification or provided with a coating.

Adsorbers of this type, as are appropriate as a second device for the removal of granulocytes and monocytes, are also the subject matter of various patent publications (see for example EP-A 0 319 961, EP-A 1 882 738, WO 2000/55621, U.S. Pat. No. 4,370,381) and are available as commercial products, for example under the trade name Adacolumn® (JIMRO Co., Ltd.).

In a preferred embodiment of the system for the treatment of blood, the at least one first device for the removal of proinflammatory mediators is a membrane filter, and the at least one second device for the removal of granulocytes and monocytes is an adsorber with a housing in whose interior space a plurality of threads is arranged with a high degree of order, as was described earlier. Especially preferably, the membrane filter is one in which the at least one semipermeable membrane is a semipermeable hollow fiber membrane. In this preferred embodiment with a membrane filter and an adsorber based on threads arranged with a high degree of order as a first and second device, the two devices are connected to each other in such a way that the retentate chamber of the first device is in fluid communication with the outer space surrounding the threads of the second device. In the especially preferred embodiment having a membrane filter with hollow fiber membranes, the lumina of the semipermeable hollow fibers that represent the retentate chamber are in fluid communication with the threads of the outer space surrounding the second device.

In the application, for these embodiments the blood to be treated flows on the first blood flow path through the blood inlet device of the membrane filter into the retentate chamber of the membrane filter, flows through the latter, wherein—by filtration—a portion of the blood to be treated passes as permeate through the semipermeable membrane, and wherein a removal of proinflammatory mediators occurs during the filtration. After flowing through the retentate chamber, the retentate exits the membrane filter via the blood outlet device, while the ultrafiltrate containing the proinflammatory mediators removed from the blood is removed from the membrane filter via the retentate outlet. After exiting the membrane filter, the retentate—that is, the blood treated in the first device—flows on the second blood flow path via the inlet device of the adsorber into the outer space surrounding the threads arranged in the adsorber, and flows over the threads on their outer side. A removal of granulocytes and monocytes from the blood flowing over the threads and to be treated is thereby accomplished via adsorption at said threads.

The blood, which is then also treated in the second device and is at this point purified of proinflammatory mediators as well as granulocytes and monocytes, then exits the adsorber via its outlet device.

It is likewise possible that the blood to be treated first flows through the first device and then through the second device. For the above preferred embodiments, this means that in the application the blood to be treated then flows on the second blood flow path via the inlet device of the adsorber into the outer space surrounding the threads arranged in the adsorber, and flows over the threads on their outer side. A removal of granulocytes and monocytes from the blood flowing over the threads and to be treated is thereby accomplished via adsorption at said threads. The blood treated in the second device and purified of granulocytes and monocytes then exits the adsorber via its outlet device.

After exiting the adsorber, this blood treated in the second device flows on the first blood flow path via the blood inlet device of the membrane filter into the retentate chamber of the membrane filter, flows through the latter, wherein—by filtration—a portion of the blood to be treated passes as permeate through the semipermeable membrane, and wherein a removal of proinflammatory mediators occurs during the filtration. After flowing through the retentate chamber, the retentate—that is, the blood also treated in the first device and at that point purified of proinflammatory mediators as well as of granulocytes and monocytes—exits the membrane filter via its blood outlet device. The permeate containing the proinflammatory mediators is removed from the membrane filter via the permeate outlet.

At least one first device for the removal of proinflammatory mediators, and at least one second device for the removal of granulocytes and monocytes, can be connected in series as separate devices of the system according to the invention, wherein—as already explained—the sequence of the devices and the sequence in which the blood to be treated passes through the devices can be adapted to the requirements of the blood treatment. In this context, however, the devices must be connected to each other in such a way that the first blood flow path of the at least one first device and the second blood flow path of the at least one second device are in fluid communication with each other, and in such a way that the blood to be treated can flow through two blood flow paths in succession (that is, it can flow through them in succession for the application for blood treatment). First device and second device can be connected via suitable hose connections, connection nozzles or adapters, for example.

First and second device can also have the form of cylinders that are directly joined to each other via an adhesive, welded, screwed or flanged joint, for example. The first device can be a hollow fiber membrane module in which the hollow fiber membranes are arranged in a cylindrical housing essentially parallel to each other in the direction of the longitudinal axis of the cylindrical housing, and the flow can enter the lumina of the hollow fiber membranes from the housing ends. The second device—also having a cylindrical housing which can contain an arrangement of threads for the adsorption of granulocytes and monocytes, a fibrous filter material or a particulate adsorption material, for example—can then be flanged at its one housing end to one of the housing ends of the first device or, for example, can be glued, welded or bolted via a cap nut to one of the housing ends of the first device.

Other integral designs of the system for blood treatment are also possible. The second device can be designed as an end cap which has a chamber filled with an adsorber material and which is bolted to one of the ends of a cylindrically formed first device. Likewise, the system for the treatment of blood can be designed in such a way that the second device is arranged concentrically in the form of a sheath around the housing of a first device having a cylindrical form, and in this way first and second devices form an integral unit.

The determination of parameters for the characterization of the semipermeable membrane of the membrane filter of the first device is based on the following measurement methods:

### Determination of the Sieve Coefficient for the Membrane Filter:

The sieve coefficients SK are determined for α₁ acidic glycoprotein (M_{W}=44,000 daltons), SK_{Gp}, albumin (M_{W}=68,000 daltons), SK_{Alb}, and for immunoglobulin G (M_{W}=180,000 daltons), SK_{IgG}. The determination of the sieve coefficients is carried out according to DIN EN ISO 8637:2014-03, in particular section 5.6.2 and FIG. 5, with freshly donated human heparin blood (10 IU/ml) on a dialysis machine (Nikkiso DBB-03), wherein the whole blood is recirculated during the measurement. The blood is set to a hematocrit of 32% and a total protein concentration of 60 g/L prior to the experiment. The determination of the hematocrit takes place using a cell counting device (e.g. ABC Pentra 60, Axon Lab Ag), and the determination of the total protein concentration takes place using a clinical analyzer (e.g. Cobas c 111, Roche Diagnostics).

The membrane filter is first rinsed with 1 liter of saline in a single pass and then with another liter of saline on a recirculating basis (20 min, 200 ml/min). In the second rinsing step, rinsing liquid is extracted via the membrane filter into the filtrate chamber (60 ml/min) by means of an external pump (MPC, Ismatec). The saline is completely displaced by the blood, and the experiment is started at 37° C. with blood flow QB=300 ml/min and a filtrate flow QF=60 ml/min (=20% of the blood flow). After 60 minutes, the specimens of blood input and output as well as a filtrate specimen are drawn, plasma is extracted from them by centrifugation, and the concentrations of al acidic glycoprotein, albumin and IgG are determined via laser nephelometry (BN ProSpec, Siemens Diagnostics). The sieve coefficient occurs as described in section 5.6.2.4 of DIN EN ISO 8637:2014-03.

### Separation Limit:

To determine the separation limit, the sieve coefficients determined according to the previously described method for α₁ acidic glycoprotein (M_{W}=44,000 daltons), SK_{Gp}, albumin (M_{W}=68,000 daltons), SK_{Alb}, and for immunoglobulin G (M_{W}=180,000 daltons), SK_{IgG}, are plotted in a diagram over the molecular weight. With integration of assumed vertices at 10,000 daltons with a sieve coefficient SK=1 and at 1 million daltons with a sieve coefficient SK=0, a compensation curve goes through the points. As a separation limit, the molecular weight is determined at a retention of 95% or a sieve coefficient SK of 0.05.

### Ultrafiltration Rate in Water UFR_{water} (Hydraulic Permeability):

### Hollow Fiber Membranes:

To determine the hydraulic permeability or the ultrafiltration rate in water UFR_{water} of the semipermeable membrane contained in the membrane filter, a test cell having a defined number of hollow fibers and length is prepared from the hollow fiber membranes to be tested. For this purpose, the hollow fibers are embedded in hot wax on both sides at their ends. After the wax hardens, the embeddings are cut out so that the lumina of the hollow fiber membranes are opened by the cut. The hollow fiber lumina in the embeddings must be checked for continuity. The length of the test cell is typically 300+/−5 mm. The number of hollow fiber membranes is generally between 160-240.

The effective surface of a test cell is defined as follows:

A=n·l·dᵢ·π·f_{dim} [m²]

where
A=effective area [m²]
n=number of capillaries
l=free length of the capillaries [mm]
dᵢ=inner diameter of the capillaries [μm]
f_{dim}=dimension factor [1·10⁻⁹ m²/(mm·μm)]

The test cell is stored before the measurement for at least 15 minutes at room temperature in deionized water (wetting) and then integrated into a test apparatus. The measurement is performed with ultrafiltrated and deionized water that is temperature-controlled to 37° C. The test cell is completely immersed in temperature-controlled water during the measurement. The test pressure upstream of the test cell is set at 200±2 mbar. The measurement is a dead-end method. The test cell is first conditioned for 900 seconds under test pressure. The actual measurement time is 60 seconds, in which the permeate produced during the measurement is volumetrically determined.

The UFR_{water} is determined according to the following formula:

$${UFR}_{Wasser} = \frac{Vw}{\frac{Δ t}{3600} \cdot A \cdot ( \frac{{P}_{O} + {P}_{E}}{2} ) \cdot {f}_{torr}} [ ml \text{/} ( h {m}^{2} mmHg ) ]$$

In which:
V_{W}=volume of water [ml] flowed through the membrane specimen during the measurement time
Δt=measurement time [s]
A=effective area [m²]
P₀=test pressure [mbar] (pressure upstream of the test cell)
P_{E}=end pressure [mbar] (pressure downstream of the test cell)
fₜₒᵣᵣ=1/1.33322; conversion [mbar] to [mmHg]

### Flat Membranes:

Disk-shaped membrane specimens having a diameter of 15 cm are punched out of the flat membrane to be tested and are clamped into a suitable specimen holder that is fluid-tight at the perimeter, such that an open measurement surface of 43.20 cm² results. The specimen holder is located in a housing through which water can flow when charged with pressure. The clamped-in membrane specimen is first allowed to soak in deionized water temperature-controlled to 37° C., and then is subjected to a flow of deionized water temperature-controlled to 37° C. under a defined pressure between 0.4 and 1.0 bar. After a lead time of 50 s until the pressure becomes constant, the water volume flowing through the membrane specimen during a measurement time of 60 seconds is gravimetrically or volumetrically determined.

The ultrafiltration rate UFR_{water} is determined according to the formula

$${UFR}_{Wasser} = 800 \cdot \frac{Vw}{Δ t \cdot A \cdot {p}_{O}} \cdot [ ml \text{/} ( h {m}^{2} mmHg ) ]$$

In which:
V_{W}=volume of water [ml] flowed through the membrane specimen during the measurement time
Δt=measurement time [min]
A=area of the membrane specimen subject to the flow (43.20 cm²)
p_{O}=set pressure during the measurement [bar]

1. A blood treatment system for treating acute inflammatory disorders caused by mediators, comprising at least one first device and at least one second device,
wherein the at least one first device has a first blood flow path for channeling blood and the at least one second device has a second blood flow path for channeling blood, and wherein the at least one first device and the at least one second device are serially connected in succession in such a way that the first blood flow path is in fluid communication with the second blood flow path,
wherein the at least one first device is a membrane filter for the removal of toxic mediators from blood, and the at least one second device is suitable for the removal of granulocytes and monocytes from blood, and
wherein the membrane filter has a housing, an interior filter space formed in the housing, and a semipermeable membrane arranged in the interior filter space that divides the interior filter space into a retentate chamber and permeate chamber,
wherein the housing has a blood inlet device and a blood outlet device that are in fluid communication with the retentate chamber, and the blood inlet device, the retentate chamber and the blood outlet device form the first blood flow path for channeling blood through the first device, and
wherein the housing furthermore has a permeate outlet for diverting permeate passing through the semipermeable membrane out of the permeate chamber, and
wherein the membrane filter has a separation characteristic such that the sieve coefficient for albumin SK_{Alb} is within the range from 0.015 to 0.35.

2. The system according to claim 1, characterized in that the semipermeable membrane is at least one semipermeable hollow fiber membrane having a wall and a lumen surrounded by the wall, and in that the retentate chamber is formed by the lumen of the at least one hollow fiber membrane.

3. The system according to claim 1, wherein the at least one hollow fiber membrane has an asymmetric pore structure across its wall, with a separating layer on the side of the wall of the hollow fiber membrane facing the lumen.

4. The system according to claim 1, characterized in that the semipermeable membrane has an ultrafiltration rate in water UFR_{water} within the range from 500 to 2000 ml/(h m² mmHg).

5. The system according to claim 1, characterized in that the membrane filter has a sieve coefficient for albumin SK_{Alb} in the presence of whole blood within the range from 0.05 to 0.3.

6. The system according to claim 1, characterized in that the membrane filter has a sieve coefficient for immunoglobulin G (IgG), SK_{IgG} within the range from 0.001 to 0.1.

7. The system according to claim 1, characterized in that the second device is a filter and/or an adsorber.

8. The system according to claim 7, characterized in that the at least one second device is a filter for the removal of granulocytes and monocytes
having a housing that has an interior space, as well as an inlet device and an outlet device that are in fluid communication with the interior space,
wherein a filter material having flow channels through which blood can flow is arranged in the interior space of the filter housing,
wherein inlet device, outlet device and the flow channels of the filter material form the second flow path in the interior space,
and wherein the filter material for the separation of granulocytes and monocytes is adapted by size exclusion and/or adsorption.

9. The system according to claim 7, characterized in that the at least one second device for the removal of granulocytes and monocytes from blood is an adsorber with a housing which has an inner side surrounding an interior space, as well as an inlet device and an outlet device,
wherein a plurality of threads is arranged in the interior space,
wherein the threads have at least one of their ends embedded in a sealing compound that is joined to the housing inner side in such a way that an outer space through which blood can flow around the threads is formed that is in fluid communication with the inlet device and the outlet device, whereby inlet device, outlet device and interior space form the second flow path,
wherein the arrangement of the threads has a high degree of order, wherein a high degree of order is understood to mean that a proportion of at least 25% of the threads is arranged along their extension direction, and
wherein the threads based on organic polymers cause formation of the complement activation product C5a in a concentration of at least 10 μg per m² of thread surface.

10. The system according to claim 9, characterized in that the threads are hollow threads with a lumen and a wall surrounding the lumen, as well as an interior, lumen-side surface and an outer surface, wherein the hollow threads are arranged in the housing in such a way that only the outer surfaces of the hollow threads are accessible to the blood flowing through the outer space, but by contrast the lumina of the hollow threads are not accessible to a fluid.

11. The system according to claim 7, characterized in that the at least one second device for the removal of granulocytes and monocytes from blood is an adsorber with a housing which has an inner side surrounding an interior space, as well as an inlet device and an outlet device,
wherein an adsorption material made up of particles is arranged in the interior space and
wherein an outer space through which blood can flow is formed around the particles of the adsorption material, which outer space is in fluid communication with the inlet device and the outlet device, whereby inlet device, outlet device and interior space form the second flow path.

12. The system according to claim 1, wherein a first device and a second device have the shape of cylinders that are directly joined to each other via an adhesive, welded, screwed or flanged joint.
